(19) United States
(12) Patent Application Publication
(10) Pub. No.: US 2019/0231858 A1
(43) Pub. Date: Aug. 1, 2019

(54) AUTOLOGOUS CELLS FOR IMMUNIZATION OF CHICKENS

(71) Applicant: Crystal Bioscience Inc., San Diego, CA (US)

(72) Inventors: Ellen COLLARINI, Oakland, CA (US); Marie-Cecile van de Lavoir, San Francisco, CA (US)

(21) Appl. No.: 16/332,332

(22) PCT Filed: Sep. 21, 2017
(86) PCT No.: PCT/US17/52818

§ 371 Date: Mar. 11, 2019

Related U.S. Application Data
(60) Provisional application No. 62/405,622, filed on Oct. 7, 2016.

(51) Int. Cl. A61K 39/00; C12N 5/077; C12N 5/0781

(52) U.S. Cl. CPC A61K 39/0005; C12N 5/0656; A61K 2039/552; A61K 2039/5156; C12N 5/0635

(57) ABSTRACT

Provided herein, among other things, is a method for immunizing a chicken with autologous cells obtained from the comb of that chicken. In some embodiments, the method may comprise obtaining a culture of primary fibroblast cells from the comb of a chicken, introducing an exogenous nucleic acid into the cells to provide for presentation or secretion of an antigen that is foreign to the chicken and immunizing the chicken with the cells.

### CROSS-REFERENCING

This application claims the benefit of U.S. provisional application Ser. No. 62/405,622, filed on Oct. 7, 2016, which application is incorporated herein by reference.

### BACKGROUND

Antibodies that recognize conformational epitopes are frequently sought in therapeutic, diagnostic and research applications. However, several classes of antigen, particularly cell membrane proteins like ion channels and G-protein coupled receptors, are difficult to prepare as antigens in a format that retains their physiological conformation. In some cases, this problem can be circumvented using membrane bound preparations such a virus-like particles and liposomes but this solution is not available for all cell membrane embedded proteins and, even if available, is time consuming. DNA immunizations can be used but typically the immune response is slow and weak. Cell based immunizations present the antigen in its physiological conformation and are attractive because most antigens can be expressed in commonly used cell lines in vitro. However, cell based immunizations are complicated for two reasons. First, the immune response that non-autologous cells generate induces a graft-vs-host reaction that quickly clears the cells from the immunized animal preventing the development of a specific immune response. The graft-vs-host reaction can be obviated using autologous cells that have a longer half life. Transfected autologous cells expressing an antigen of interest therefore elicit an immune response that is sustained for an extended period. Secondly, most antibodies produced in response to non-autologous cells recognize endogenous antigens that are expressed on the surface of the non-autologous cells and not shared by the host animal. For example, immunization of chickens with CHO cells expressing an antigen of interest induces an immune response that is primarily aimed at surface proteins on CHO cells. Isolating those rare antibodies to the target of interest from the overwhelming population of antibodies directed at CHO cell surface antigens greatly limits the utility of this approach. Production of antibodies to off-target antigens on the cell surface is reduced by using cells derived from the same species. For example, DT40 cells, which are ALV transformed B cells from the SC strain of chickens, can be used to immunize chickens from the same strain. Differences between individual chickens, however, induce a plethora of anti-haplotype antibodies against polymorphic proteins. Anti-haplotype antibodies are eliminated when autologous cells expressing the target of interest are used.

### SUMMARY

Provided herein, among other things, is a method for immunizing a chicken with autologous cells that are derived from the comb of that chicken. In some embodiments, the method may comprise obtaining a culture of primary fibroblast cells from the comb of a chicken, introducing an exogenous nucleic acid into the cells to provide for presentation or secretion of an antigen that is foreign to the chicken, and immunizing the chicken with the cells.

Removal of the comb (referred to as “dubbing” in the poultry industry) is routinely practiced to prevent injury to this appendage in sexually mature birds. Removal of the comb is executed in seconds without any collateral damage or trauma to the bird. Primary fibroblasts can be prepared from each comb using cell culture techniques to produce large populations of autologous fibroblasts within a few weeks. The fibroblasts can then transfected with the gene of interest and express the antigen in its native conformation on the cell surface or as a soluble protein. The population of transfected fibroblasts can be injected intravenously into the bird from which the comb originated to induce a specific antigenic response to the target.

### BRIEF DESCRIPTION OF THE DRAWINGS

Some aspects of the preset invention may be best understood from the following detailed description when read in conjunction with the accompanying drawings. It is emphasized that, according to common practice, the various features of the drawings are not to scale. Indeed, the dimensions of the various features are arbitrarily expanded or reduced for clarity. Included in the drawings are the following figures.

FIG. 1 Left: Bright field image of primary fibroblasts isolated from chicken combs at hatching. Right: Same image showing GFP expression.

FIG. 2 shows the results of flow cytometry analysis of fibroblasts labeled with an anti-FLAG antibody followed by a donkey-anti-mouse-APC secondary antibody.

FIG. 3 shows 3 shows that the pre-immune plasma does not bind either GFP-positive or GFP-negative cells, whereas plasma from draw 1 or draw 2 after immunization did not bind GFP-negative cells but did bind the cell surface of the GFP-positive cells. This data indicates that there are CCR5-specific antibodies in the chicken serum.

### DEFINITIONS

The terms “antibody” and “immunoglobulin” are used interchangeably herein. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, polyclonal antibodies, monoclonal antibodies and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986),).

It is understood that the humanized antibodies designed and produced by the present method may have additional conservative amino acid substitutions which may have substantially no effect on antigen binding or other antibody functions. By conservative substitutions is intended combinations such as gly, ala; val, ile, leu; asp, glu; asn, gln; ser, thr; lys, arg; and phe, tyr. In some embodiments, an antibody may be humanized.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Determining the presence of” includes determining the amount of something present, as well as determining whether it is present or absent.

The terms “polypeptide” and “protein”, used interchangeably herein, refer to a polymeric form of amino acids of any length, i.e. greater than 2 amino acids, greater than about 5 amino acids, greater than about 10 amino acids, greater than about 20 amino acids, greater than about 50 amino acids, greater than about 100 amino acids, greater than about 200 amino acids, greater than about 500 amino acids, greater than about 1000 amino acids, greater than about 2000 amino acids, usually not greater than about 10,000 amino acids, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Also included by these terms are polypeptides that are post-translationally modified in a cell, e.g., glycosylated, cleaved, secreted, prenylated, carboxylated, phosphorylated, etc, and polypeptides with secondary or tertiary structure, and polypeptides that are covalently or non-covalently bound to other moieties, e.g., other polypeptides, atoms, cofactors, etc.

A “coding sequence” or a sequence that “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells, which can be accomplished by genomic integration of all or a portion of the vector, or transient or inheritable maintenance of the vector as an extrachromosomal element. Thus, the term includes cloning, and expression vehicles, as well as integrating vectors.

An “expression cassette” comprises any nucleic acid construct capable of directing the expression of a gene/coding sequence of interest, which is operably linked to a promoter of the expression cassette. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

A first polynucleotide is “derived from” a second polynucleotide if it has the same or substantially the same nucleotide sequence as a region of the second polynucleotide, its cDNA, complements thereof, or if it displays sequence identity as described above. A first polynucleotide may be derived from a second polynucleotide if the first polynucleotide is used as a template for, e.g. amplification of the second polynucleotide.

The term “introducing” in the context of inserting a nucleic acid sequence into a cell, includes “transfection” and “transformation” and all other methods of introducing a nucleic acid into a cell, where the nucleic acid sequence may be incorporated into the genome of the cell (e.g., chromosome, plasmid, plastid, or mitochondrial DNA) or converted into an autonomous replicon, or transiently expressed.

The term “introduced” in the context of inserting a nucleic acid sequence into a cell, means “transfection”, or ‘transformation”, or “transduction” and includes reference to the incorporation of a nucleic acid sequence into a eukaryotic or prokaryotic cell wherein the nucleic acid sequence may be present in the cell transiently or may be incorporated into the genome of the cell (e.g., chromosome, plasmid, plastid, or mitochondrial DNA), converted into an autonomous replicon.

The term “plurality” refers to at least 2, at least 5, at least 10, at least 20, at least 50, at least 100, at least 200, at least 500, at least 1000, at least 2000, at least 5000, or at least 10,000 or at least 50,000 or more. In certain cases, a plurality includes at least 10 to 50. In other embodiments, a plurality may be at least 50 to 1,000.

The term “primary fibroblast cells” refers to cells that are cultured directly from a subject. Such cells undergo senescence and stop dividing after a certain number of population doublings while generally retaining their viability.

The term “derived from”, in the context of cells that are derived from a tissue, refers to cells that have descended from an original population of cells in the tissue, via cell culture.

The term “comb” refers to the fleshy growth or crest on the top of the head of a chickens. Chicken combs are most commonly red (but may be black or dark purple in certain breeds).

The term “present” in the context of presenting an antigen refers to the location of an antigen on the exterior surface of a cell. In some embodiments, this can be done by linking the antigen to a transmembrane region that tethers the antigen to the surface of the cell to “display” the antigen on the outside of the cell. In other cases, the antigen may be a transmembrane protein (e.g., a integral membrane protein) and, as such, the cell may present the antigen because it is embedded in the plasma membrane and parts of the antigen (e.g., the exterior loops of the antigen) are exposed.

The term “foreign to the chicken” refers to an antigen that is not expressed by the chicken. An antigen that is foreign to a chicken can be from another species (e.g., from a mammal such as a human or mouse). Antigens that are foreign to a chicken possess epitopes that are not present in the chicken.

The term “tethered to the surface of the cells” refers to linking an antigen to a transmembrane region that anchors the antigen to the surface of the cell. A tether can be placed at the N- or C-terminal end of a soluble protein to anchor it a cell.

The term “immunizing the chicken with the cells” refers to a protocol in which cells are administered to a chicken in order to initiate an immune response from the chicken. In some cases, immunizing may require multiple administrations (e.g., one or more “booster” shots). Protocols for immunizing chickens with an antigen are well known.

The term “exogenous nucleic acid” refers to a nucleic acid that is recombinant, i.e., that contains nucleic acid elements that are joined together in a way that is not natural, and that is introduced into the cell from the outside of the cell.

The term “provides for”, in the context of a construct that provides for presentation or secretion of a protein refers to a construct that has at least the coding sequence for a protein and in some cases has all of the necessary regulatory elements (e.g., promoter, terminator, untranslated regions, etc.) as well as any optional coding sequences (e.g., secretion signal, transmembrane tether, etc.) that facilitate expression and tethering or secretion of a protein to or from the surface of a cell.

The term “autologous” means from the same individual.

A cell is “derived from” a host if the cell was obtained from the host. The progeny of a progenitor cell are derived from the same host as a progenitor cell.

As used herein, the term “isolated”, with respect to a cell, refers to a cell that is cultured in vitro.

As used herein, the term “culture”, with respect to a culture of cells, refers to a population of cells that: a) are grown in vitro in a synthetic growth medium and b) are capable of undergoing or have undergone a limited number of cell divisions in the medium.

The phrase “in a medium” is intended to encompass growth on top of the medium as well as growth in the medium.

As used herein, a “synthetic” growth medium refers to man-made medium that is not a living, multicellular organism. The term “synthetic growth medium” explicitly excludes living animals.

The term “transmembrane receptor” refers to any integral membrane protein whose activity can be modulated by ligand binding or a conformational change. G protein-coupled, ion channel-linked, enzyme-linked, carrier transport proteins, PAQR and sigma receptors are types of transmembrane receptor. G protein-coupled receptors (GPCRs) possess seven transmembrane alpha helices as described in greater detail herein. When a GPCR is activated, the GPCR activates an associated G-protein that in turn activates intracellular signaling cascades. Ion channel-linked receptors (i.e., ligand gated ion channels) are also known as ionotropic receptors.

Binding of a ligand to such an ion channel results in opening of the channel to increase ion flow through the channel or closing to decrease ion flow. Enzyme-linked receptors (also known as catalytic receptors) are receptors in which activation by binding of an extracellular ligand triggers enzymatic activity on the intracellular side of the protein. Carrier proteins couple the transport of ions, small molecules, and macromolecules across the cellular membrane to conformational changes in the receptor through passive, ‘facilitated diffusion’ or through active transport mechanisms requiring an electrochemical gradient or adenosine trisphosphate dependent processes.

“G-protein coupled receptors” or “GPCRs” are polypeptides that share a common structural motif, having seven regions of between 22 to 24 hydrophobic amino acids that form seven alpha helices, each of which spans a membrane. Each span is identified by number, i.e., transmembrane-1 (TM1), transmembrane-2 (TM2), etc. The transmembrane helices are joined by regions of amino acids between transmembrane-2 and transmembrane-3, transmembrane-4 and transmembrane-5, and transmembrane-6 and transmembrane-7 on the exterior, or “extracellular” side, of the cell membrane, referred to as “extracellular” regions 1, 2 and 3 (EC1, EC2 and EC3), respectively. The transmembrane helices are also joined by regions of amino acids between transmembrane-1 and transmembrane-2, transmembrane-3 and transmembrane-4, and transmembrane-5 and transmembrane-6 on the interior, or “intracellular” side, of the cell membrane, referred to as “intracellular” regions 1, 2 and 3 (IC1, IC2 and IC3), respectively. The “carboxy” (“C”) terminus of the receptor lies in the intracellular space within the cell, and the “amino” (“N”) terminus of the receptor lies in the extracellular space outside of the cell. GPCR structure and classification is generally well known in the art, and further discussion of GPCRs may be found in Probst, DNA Cell Biol. 1992 11:1-20; Marchese et al Genomics 23: 609-618, 1994; and the following books: Jurgen Wess (Ed) Structure-Function Analysis of G Protein-Coupled Receptors published by Wiley-Liss (1st edition; Oct. 15, 1999); Kevin R. Lynch (Ed) Identification and Expression of G Protein-Coupled Receptors published by John Wiley & Sons (March 1998) and Tatsuya Haga (Ed), G Protein-Coupled Receptors, published by CRC Press (Sep. 24, 1999); and Steve Watson (Ed) G-Protein Linked Receptor Factsbook, published by Academic Press (1st edition; 1994).

Further definitions may be elsewhere in this disclosure.

### DESCRIPTION OF EXEMPLARY EMBODIMENTS

Before the present subject invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a cell” includes a plurality of cells and reference to “a candidate agent” includes reference to one or more candidate agents and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As noted above, a culture of primary fibroblast cells derived from the comb of a chicken is provided. In some embodiments, the fibroblast cells in the culture present or secrete an antigen that is foreign to the chicken, meaning that the cells contain a construct that encodes a protein that is foreign to the chicken (e.g., a non-chicken protein such as a protein from another species, e.g., a mammal, or a synthetic sequence), and the foreign protein is secreted from, tethered to, or integral with the cell surface. In these embodiments, the antigen can be secreted from the cells, by adding a secretion signal to the protein. Alternatively, the construct may provide for targeting of the antigen to the surface of the host cell by producing an antigen that is linked to a cell surface targeting polypeptide. In such embodiments, the coding sequence for the antigen may be operably linked to a coding sequence for a targeting polypeptide-encoding nucleic acid in the construct, and transcription and subsequent translation of the coding sequences provides for production of a fusion protein containing the antigen and the cell surface targeting polypeptide. As such, the expression cassette can provide for targeting of an antigen to the surface of a host cell, which antigen is not usually presented on the surface of the host cell. Suitable cell surface targeting polypeptides and their encoding nucleic acid sequences may be those of, for example, transmembrane serine threonine or tyrosine kinase receptors. Suitable cell surface targeting signals and their encoding nucleic acid sequences include receptor transmembrane domains, such as the epidermal growth factor receptor (EGFR) transmembrane domain (Ullrich, A. et al. Nature 309: 418-425 (1984)). In some embodiments, the cell surface targeting sequence may be from chicken. Examples of strategies for targeting of polypeptides in a cell or protein secretion may be found in U.S. Pat. No. 6,455,247. In certain embodiments, the construct may contain a recombinant polynucleotide containing an expression cassette, i.e., a promoter, a polynucleotide encoding a protein, and a transcriptional terminator, where the expression cassette is sufficient for the production of the protein by a chicken. The recombinant nucleic acid may integrate into the genome of the host cell, or it may be present in a vector that replicates autonomously from the genome. In certain embodiments, the polynucleotide encoding the antigen may be codon optimized for expression of the protein in the chicken cell.

In some embodiments, the antigen may be an integral membrane protein and, as such may have one or more transmembrane regions. In some embodiments, the protein may be any type of transmembrane receptor, such as, e.g., a GPCR, a transporter, or an ion channel. In particular embodiments, the transmembrane receptor is a GPCR. Any known GPCR can be used in the method. A disclosure of the sequences and phylogenetic relationships between 277 GPCRs is provided in Joost et al. (Genome Biol. 2002 3:RESEARCH0063, the entire contents of which is incorporated by reference), and the phylogenetic relationships between 367 human and 392 mouse GPCRs is provided in Vassilatis et al. (Proc Natl Acad Sci 2003 100:4903-8 and the primalinc website, each of which is hereby incorporated by reference in its entirely). GPCR families are also described in Fredriksson et al (Mol. Pharmacol. 2003 63, 1256-72). GPCRs include purinergic receptors, vitamin receptors, lipid receptors, peptide hormone receptors, protein receptors, non-hormone peptide receptors, non-peptide hormone receptors, polypeptide receptors, protease receptors, receptors for sensory signal mediator, and biogenic amine receptors.

It is recognized that both native (naturally occurring) and altered native (non-naturally occurring) proteins can be used as an antigen herein. In certain embodiments, therefore, an altered native GPCR (e.g. a native GPCR that is altered by an amino acid substitution, deletion and/or insertion) such that it binds the same ligand as a corresponding native GPCR, and/or couples to a G-protein as a result of the binding. In certain cases, a GPCR employed herein may have an amino acid sequence that is at least 80% identical to, e.g., at least 90% identical, at least 85% identical, at least 90% identical, at least 95% identical, or at least 98% identical, to at least the heptahelical domain of a naturally occurring GPCR. A GPCR employed herein may optionally contain the C-terminal domain of a GPCR. In certain embodiments, a native GPCR may be “trimmed back” from its N-terminus and/or its C-terminus to leave its heptahelical domain.

Exemplary GPCRs that can be used in the subject include, but are not limited to 5-HT1A, 5-HT1B, 5-HT1D, 5-ht1e, 5-HT1F, 5-HT2A, 5-HT2B, 5-HT2C, 5-HT4, 5-ht5a, 5-HT6, 5-HT7, M1, M2, M3, M4, M5, A1, A2A, A2B, A3, alpha 1A-adrenoceptor, alpha 1B-adrenoceptor, alpha 1D-adrenoceptor, alpha 2A-adrenoceptor, alpha 2B-adrenoceptor, alpha 2C-adrenoceptor, beta 1-adrenoceptor, beta 2-adrenoceptor, beta 3-adrenoceptor, C(3a, C5a, C5L2, AT1, AT2, APJ, GPBA, BB1, BB2, BB3, B1, B2, CB1, CB, B2 CCR1, CCR2, CCR3, CCR4, CCR5S, CCR6, CCR7, CCR8, CCR9, CCR10, CXCR1, CXCR2, CXCR3, CXCR4, CXCR5, CXCR6, CXCR7, CX3CR1, XCR1, CCK1, CCK2, D1, D2, D3, D.sub.4, D5, ETA, ETB, GPER, FPR1, FPR2/ALX, FPR3, FFA1, FFA2, FFA3, GPR42, GAL1, GAL2, GAL3, ghrelin, FSH, LH, TSH, GnRH, CGnRH2, H1, H2, H3, H4, HCA1, HCA2, HCA3, kisspeptin, BLT1, BLT2, CysLT1, CysLT2, OXE, FPR2/ALX, LPA1, LPA2, LPA3, LPA4, LPA5, S1P1, S1P2, S1P3, S1P4, S1P5, MCH1, MCH2, MC1, MC2, MC3, MC4, MC5, MT1, MT2, motilin, NMU1, NMU2, NPFF1, NPFF2, NPS, NPBW1, NPBW2, Y1, Y2, Y4, Y5, NTS1, NTS2, delta, kappa, mu, NOP, OX1, OX2, P2Y1, P2Y2, P2Y4, P2Y6, P2Y11, P2Y12, P2Y13, P2Y14, QRFP, PAF, PKR1, PKR2, PRRP, DP1, DP2, EP1, EP2, EP3, EP4, FP, IP1, TP, PAR1, PAR2, PAR3, PAR4, RXFP1, RXFP2, RXFP3, RXFP4, sst1, sst2, sst3, sst4, sst5, NK1, NK2, NK3, TRH1, TA1, UT, V1A, V1B, V2, OT, CCRL2, CMKLR1, GPR1, GPR3, GPR4, GPR6, GPR12, GPR15, GPR17, GPR18, GPR19, GPR20, GPR21, GPR22, GPR25, GPR26, GPR27, GPR31, GPR32, GPR33, GPR34, GPR35, GPR37, GPR37L1, GPR39, GPR42, GPR45, GPR50, GPR52, GPR55, GPR61, GPR62, GPR63, GPR65, GPR68, GPR75, GPR78, GPR79, GPR82, GPR83, GPR84, GPR85, GPR87, GPR88, GPR101, GPR119, GPR120, GPR132, GPR135, GPR139, GPR141, GPR142, GPR146, GPR148, GPR149, GPR1500, GPR11, GPR51, GPR152, GPR153, GPR160, GPR161, GPR162, GPR171, GPR173, GPR174, GPR176, GPR182, GPR183, LGR4, LGR5, LGR6, LPAR6, MAS1, MAS1L, MRGPRD, MRGPRE, MRGPRF, MRGPRG, MRGPRX1, MRGPRX2, MRGPRX3, MRGPRX4, OPN3, OPN5, OXGR1, P2RY8, P2RY10, SUCNR1, TAAR2, TAAR3, TAAR4, TAAR5, TAAR6, TAAR8, TAAR9, CCPB2, CCRL1, FY, CT, calcitonin receptor-like, CRF1, CRF2, GHRH, GIP, GLP-1, GLP-2, glucagon, secretin, PTH1, PTH2, PAC1, VPAC1, VPAC2, BAI1, BAI2, BAI3, CD97, CELSR1, CELSR2, CELSR3, ELTD1, EMR1, EMR2, EMR3, EMR4P, GPR56, GPR64, GPR97, GPR98, GPR110, GPR111, GPR112, GPR113, GPR114, GPR115, GPR116, GPR123, GPR124, GPR125, GPR126, GPR128, GPR133, GPR143, GPR144, GPR157, LPHN1, LPHN2, LPHN3, CaS, GPRC6, GABAB1, GABAB2, mGlu1, mGlu2, mGlu3, mGlu4, mGlu5, mGlu6, mGlu7, mGlu8, GPR156, GPR158, GPR179, GPRC5A, GPRC5B, GPRC5C, GPRC5D, frizzled, FZD1, FZD2, FZD3, FZD4, FZD5, FZD6, FZD7, FZD8, FZD9, FZD10, SMO. In certain embodiments, the method may use the β2-adrenergic receptor (β 2AR), the A2A-Adenosine Receptor (A₂A), S1P1, an opioid receptor (OLR), e.g., NOP1, a chemokine receptor, e.g., CXCR3 or CCR5 (CCR5), GLP1R, PTHR1, LPA1, LPA2, LPA3, S1P2, S1P3, S1P4, or S1P5. The GPCR used may be of any class, e.g., Class A (rhodopsin-like); Class B (secretin-like); Class C (metabotropic glutamate/pheromone); cAMP receptors vomeronasal receptors (V1R and V3R); and taste receptors T2R. GPCRs to be evaluated include, but are not limited to, a class A GPCR, a class B GPCR, a class C GPCR, a class D GPCR, a class E GPCR, and a class F GPCR, including orthologs from any mammalian species, e.g., human or mouse, etc.

In some embodiments, the antigen may have a particular property, e.g., it may be differentially expressed in a disease or condition or expressed in a certain tissue, expressed at a certain time during normal or abnormal development, or encode polypeptides with certain activity, e.g., an activity that is associated with a disease or condition.

The above-described culture may be made by obtaining a culture of primary fibroblast cells from the comb of a chicken (typically a newly hatched chicken), e.g., using the method described below, and introducing an exogenous nucleic acid into the cells, wherein the exogenous nucleic acid provides for presentation or secretion of an antigen that is foreign to the chicken. Fibroblasts can be obtained from various tissues by cutting the tissue into small pieces (see, e.g., Gandrillon, et al., Cell 1987 49:687-697) and placing them in culture. The process can also include adding enzymatic dissociation reagents, such as trypsin, collagenase, dispase, hyaluronidase and others, to improve recovery by digesting connective tissue that hold the cells together (e.g., Tuan, et al., 2008 Am J Pathol 173:1311-1325; Hernandez and Brown, 2010 Curr. Protoc. Microbiol. 17:A.41.1-A.41.8). A general discussion on tissue dissociation can be found in the Worthington Tissue Dissociation Guide, which is posted to the Worthington-Biochem website. In one example one can use trypsin to aid in recovering fibroblasts from combs. Other dissociation reagents may be used instead. There are numerous medium formulations that can be used to culture fibroblasts, such as Medium 199 (see, Gandrillon, et al., 1987, supra), DMEM (Froble, et al., 1979 Mol. Cell. Endocrinol. 13:35-45), EMEM (Hernandez and Brown, 2010), DMEM/F12 (Seluanov et al., 2010 J. Vis. Exp. 44:2033), and others, with various supplements such as fetal calf serum, tryptone, and/or chicken serum (Gandrillon, et al, supra, Froble, et al, 1979, supra). In one example, one can use DMEM supplemented with fetal calf serum and chicken serum.

Nucleic acids may be introduced into a cell using a variety of methods, including viral infection, transfection, conjugation, protoplast fusion, electroporation, particle gun technology, calcium phosphate precipitation, direct microinjection, viral vector delivery, and the like. There are numerous reagents for transfecting cells, such as Lipofectamine, Optifect, DMRIE-C (all from Life Technologies), Viromer (Origene), polyethylenimine-based reagents (e.g., PEIpro from Polyplus), and others. The choice of method is generally dependent on the type of cell being transformed and the circumstances under which the transformation is taking place (i.e., in vitro). A general discussion of these methods can be found in Ausubel, et al, Short Protocols in Molecular Biology, 3rd ed., Wiley & Sons, 1995. In some embodiments, the cells that contain the exogenous nucleic acid may be selected, e.g., by FACS or using selectable marker, thereby producing a culture in which at least 10%, at least 30%, at least 50% or at least 80% of the cells contain the exogenous nucleic acid. In some embodiments, the cells may be cultured after introduction of the exogenous so that the culture contains at least 10³, at least 10⁴, at least 10⁵ or at least 10⁶ of the cells.

In particular embodiments, the culture may be cryogenically frozen and, as such, may be in a frozen state at about −80° C. or below (e.g., in liquid nitrogen). In these embodiments, the culture may contain cells that are suspended in a liquid that contains a cryoprotectant, e.g., glycerol, methanol or DMSO (e.g., 5%-15% DMSO), for example. A cryovial containing the culture in cryoprotective agent at room temperature may be inserted into a special freezing container, e.g., a Nalgene “Mr Frosty” container, which has been pre-chilled to refrigerator temperature. The freezing container may then be placed into a −70° C. freezer for a period of time. Then the cryovial may quickly removed from the freezing container, placed into a storage container, and plunged into liquid nitrogen for indefinite storage. After a period of time, the cells can be thawed and used.

The culture of cells, because it can be readily made from a comb of a chicken without sacrificing the chicken can be used to immunize the chicken from which they are derived (i.e., the same individual chicken). In these methods, the host cells of the cell culture are “autologous” to the chicken, meaning that they are from the same individual. In some embodiments, this method may comprise making a culture of primary fibroblast cells as described above using fibroblasts from the comb of a chicken, and immunizing the chicken with the cells, e.g., live cells. The cells may be administered using any convenient method, e.g., by intravenous injection into the wing vein, breast muscle or subcutaneously. The cells may be live in the bloodstream for some period of time (e.g., hours, days or weeks) and, as such, the host check should mount an immune response to epitopes that appear to be foreign to the chicken, thereby producing antibodies to the foreign antigen. One some embodiments, the cells may be combined with a high affinity agonist or antagonist of the foreign antigen, thereby locking the antigen in an active or inactive state prior to immunization.

Methods of immunizing animals, including the adjuvants used, booster schedules, sites of injection, etc. are well understood in the art, e.g., Harlow et al. (Antibodies: A Laboratory Manual, First Edition (1988) Cold spring Harbor, N.Y., and Harlow, supra), and administration of living cells to animals has been described for several mammals and birds, e.g., McKenzie et al (Oncogene 4:543-8, 1989), Scuderi et al (Med. Oncol. Tumor Pharmacother 2:233-42, 1985), Roth et al (Surgery 96:264-72, 1984) and Drebin et al (Nature 312:545-8, 1984). In some embodiments a chicken may be immunized with at least 10⁴, at least 10⁵, or at least 10⁶, cells.

After a chicken has been immunized, the chicken will mount an immune response against foreign antigen, and the blood of an immunized animal will normally contain polyclonal antisera that bind to the antigen (e.g., by ELISA, western blot, etc.). Binding affinities of the polyclonal antisera to the antigens may vary between antigen, but will generally be in the range of at least about 10⁶M⁻¹ at least about 10⁷M⁻¹, at least about 10⁸ M⁻¹, or at least about 10⁹ M⁻¹ to 10¹⁰ M⁻¹) for the cells. Polyclonal antisera can be harvested from the immunized chicken using methods well known in the art and used in the subject methods.

In certain embodiments, B cells that produce monoclonal antibodies against the foreign antigen can be isolated from the chicken. These cells may be from the blood, spleen or bursa, for example. In certain embodiments, the cells may be fused to any convenient fusion partner and screened using conventional methods.

The methods and compositions described herein find particular use in producing antibodies that recognize conformational epitopes in proteins that are difficult to express in other expression systems and/or that are insoluble (e.g., GPCRs, ion channels, etc). When the foreign protein is expressed in a chicken cell, the protein should fold into its native conformation and, after immunization, any conformational epitopes should be presented to the immune system of the chicken, resulting in antibodies that recognize a conformational epitope.

### EMBODIMENTS

1. A culture of primary fibroblast cells derived from the comb of a chicken is provided. In some embodiments, the fibroblast cells in the culture may present or secrete an antigen that is foreign to the chicken.
2. The culture of embodiment 1, wherein the antigen is secreted from the cells.
3. The culture of embodiment 1, wherein the antigen is presented on the surface of the cells.
4. The culture of embodiment 3, wherein the antigen is tethered to the surface of the cells.
5. The culture of embodiment 3, wherein the antigen is an integral membrane protein.
6. The culture of embodiment 4, wherein the antigen is a cell surface receptor.
7. The culture of embodiment 5, wherein the antigen is a G protein-coupled receptor.
8. The culture of embodiment aim 6, wherein the antigen is an ion channel.
9. A method for autologous immunization of a chicken, comprising: making a culture of primary fibroblast cells of any of embodiment 1-8 using fibroblasts from the comb of a chicken and immunizing the chicken with the cells.
10. The method of embodiment 9, wherein the method comprises: (a) obtaining a culture of primary fibroblast cells from the comb of a chicken; (b) introducing an exogenous nucleic acid into the cells, wherein the exogenous nucleic acid provides for presentation or secretion of an antigen that is foreign to the chicken; and (c) immunizing the chicken with the cells.
11. The method of any of embodiment 9-10, wherein the chicken is a newly hatched chicken.
12. The method of any of embodiment 9-11, further comprising obtaining polyclonal antibodies from the chicken after immunization.
13. The method of any of embodiments 9-12, further comprising obtaining B cells from the chicken after immunization.
14. An isolated B cell or hybridoma thereof, from a chicken made by the method of any of embodiments 9-13.
15. A culture of primary fibroblast cells derived from the comb of a chicken.

### EXAMPLES

The following examples are provided in order to demonstrate and further illustrate certain embodiments and aspects of the present invention and are not to be construed as limiting the scope thereof.

### Example 1

### Harvesting Combs from Newly Hatched Chicks

At hatch chicks were individually tagged and a small blood sample is taken to establish the genotype of the individual. Within a few days following hatch, the comb of chicks with the desired genotype was wiped with an alcohol containing wipe, removed by cutting with scissors and placed in an Eppendorf tube containing PBS.

### Example 2

### Establishing a Culture of Autologous Primary Fibroblasts

The comb tissue was cut into small pieces using a razor blade. The pieces were transferred using phosphate buffered saline (PBS) to a tube and pelleted. The tissue was resuspended in 300 ul 0.25% trypsin/0.1% EDTA for 20 min. The tissue was triturated, and 1 ml of DMEM, supplemented with Glutamax, 7.5% fetal calf serum, 2.5% irradiated chicken serum, and 1× Pen/Strep, was added. The tissue was centrifuged, resuspended in 1 ml of the same medium, and placed in a single well of a 12 well dish. The cultures were incubated until the wells were near confluent with cells. The cells were trypsinized and re-plated into 6 well dishes. When the 6 well was confluent, the cells were trypsinized and aliquots were cryopreserved in medium+10% DMSO. When needed, the cells were thawed in the same medium without Pen/Strep and expanded and cryopreserved as needed. Upon thawing, the cell population expanded to at least 3×10⁸ within 12 days.

### Example 3

### Transient Transfection of Autologous Primary Fibroblasts

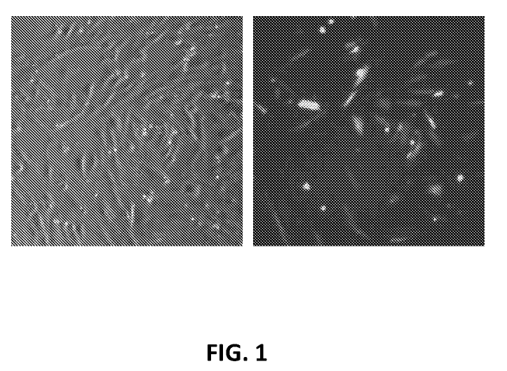

There are numerous reagents for transfecting cells, such as Lipofectamine, Optifect, DMRIE-C (all from Life Technologies), Viromer (Origene), polyethylenimine-based reagents (e.g., PEIpro from Polyplus), and others. In this example Optifect was used. The cells were trypsinized, counted, and plated at a density of 1e4/cm2. The next day the DNA encoding GFP was added to Optifect at a ratio 1 ug/2 ul. The mixture was incubated for 20 min, then added to the cells. The cells were incubated for 2-3 days to allow for maximum expression of the exogenous protein. The proportion of cells expressing GFP expression was quantitated by flow cytometry, and approximately 40% of the cells expressed this construct (see Table 1 and FIG. 1).

TABLE 1
Proportion of cells expressing
GFP 2 days post transfection

| Sample | cell count | % GFP |
|---|---|---|
| control | 30000 | 0 |
| 1 ug:1 ul Optifect | 30000 | 20.2 |
| 1 ug:2 ul Optifect | 30000 | 42.7 |
| 1 ug:3 ul Optifect | 14627 | 40.9 |
| 1 ug:4 ul Optifect | 4817 | 36.9 |

### Example 4

### Transient Transfection of Autologous Primary Fibroblasts with DNA Encoding a Membrane Protein

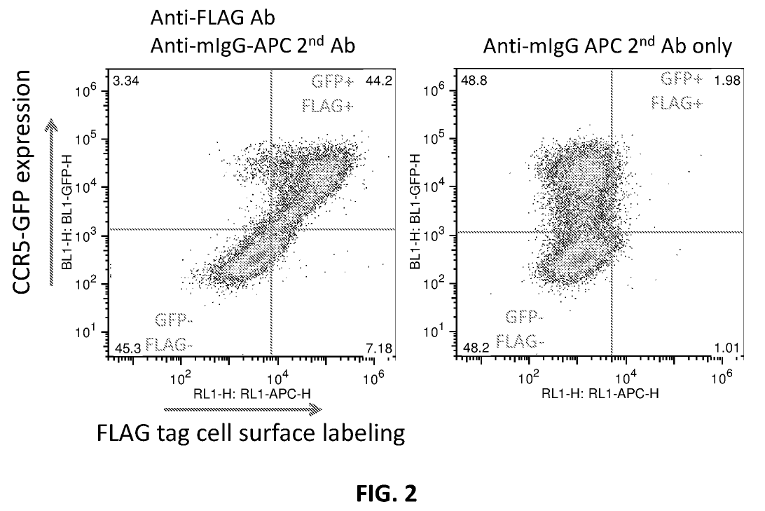

In this example, 7.3e5 chicken fibroblasts were plated in a T25 flask. The next day the medium was changed, and 2-4 hours later the cells were transfected with a construct encoding the G-protein coupled receptor CCR5 as a GFP fusion protein and with an extracellular FLAG tag. In this example, 119 ul Viromer transfection buffer was added to 6.7 ul Viromer transfection reagent, and then 12.5 ug DNA diluted into 1.13 ml Viromer transfection buffer was added. The mix was incubated for 15 minutes then added to the cells. The next day cells were lifted from the flask using a mild enzymatic solution (e.g. diluted Accutase, Life Technologies), labeled with an anti-FLAG antibody followed by a donkey-anti-mouse-APC secondary antibody, and analyzed by flow cytometry (see FIG. 2). The graph on the left shows 44.2% of the cells expressed GFP and were positive for FLAG on the cell surface (upper right quadrant), while the GFP-negative cells were also negative for FLAG (lower left quadrant). This indicates that 44.2% of the cells have been successfully transfected, and that CCR5-FLAG is expressed at the cell surface of the transfected cells. The graph on the left shows cells labeled with only the secondary antibody as a control.

### Example 5

### Injection of Autologous Primary Fibroblasts into Chickens

In this example, autologous cells were plated at approximately 2e6 cells in each of two T75 flasks, and transfected with the CCR5 construct as in Example 4, scaling up the volumes by a factor of 3 because of the larger flask size. The next day transfection efficiency was determined by observing GFP expression at the microscope (approximately 40% of the cells were GFP positive). The cells were lifted from the culture vessels using a mild enzymatic solution, washed twice in PBS, and re-suspended in 0.5 ml PBS. This cell suspension was injected intravenously when the chicken from which the comb cells were derived reached 6-8 weeks of age. The transfection and injection were repeated 4 times, every two weeks, until the desired immune response had been obtained.

### Example 6

### Development of the In Vivo Immune Response to the Target

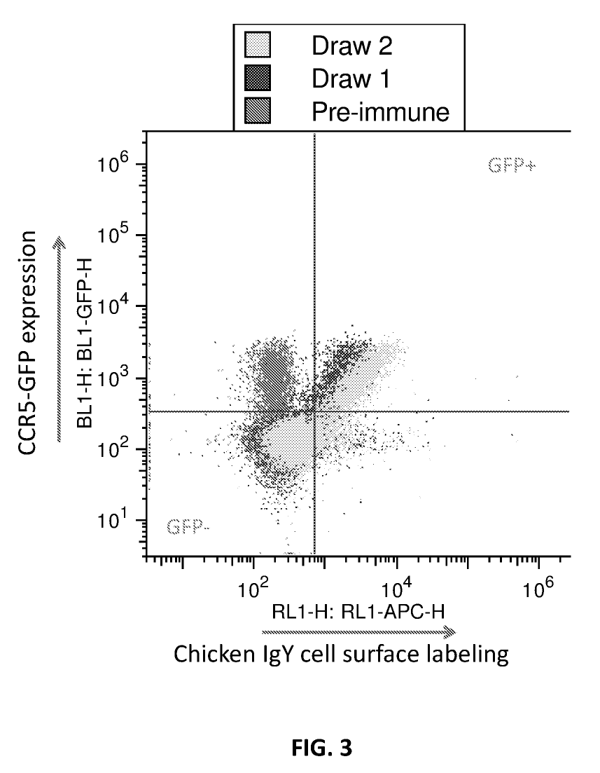

Starting one week after the second injection, 0.5 ml of blood was drawn from the wing vein of the immunized animal using a 23-gauge needle to evaluate serum titer. The week after each subsequent immunization another blood sample was drawn. For each draw, the blood was centrifuged and plasma was recovered and frozen in aliquots. Plasma from the chicken taken before immunization (pre-immune), after 2 injections (draw 1), and after 3 injections (draw 2) of autologous transfected cells was incubated with EXPI293 cells (Life Technologies) that had been transfected with the CCR5-GFP DNA construct, followed by a goat anti-chicken IgY-Alexa647 secondary antibody. FIG. 3 shows that the pre-immune plasma (red population) did not bind either GFP-positive or GFP-negative cells. Plasma from draw 1 (blue population) or draw 2 (green population) after immunization did not bind GFP-negative cells, but did bind the cell surface of the GFP-positive cells, indicating that there are CCR5-specific antibodies in the chicken serum. The signal strength increased from draw 1 to draw 2, indicating that continued immunization results in a more robust response.

### Example 7

### Isolation of Monoclonal Antibodies Recognizing the Target

After several immunizations, and when the animal had developed a good immune response, the bird was euthanized according to approved procedures. The spleen was taken and processed to recover the lymphocytes expressing a specific antibody against the protein of interest using Crystal's proprietary GEM assay. Antibody genes were recovered from selected lymphocytes and reconstituted as recombinant antibodies.

1. A culture of primary fibroblast cells derived from the comb of a chicken, wherein the fibroblast cells in the culture present or secrete an antigen that is foreign to the chicken.

2. The culture of claim 1, wherein the antigen is secreted from the cells.

3. The culture of claim 1, wherein the antigen is presented on the surface of the cells.

4. The culture of claim 3, wherein the antigen is tethered to the surface of the cells.

5. The culture of claim 3, wherein the antigen is an integral membrane protein.

6. The culture of claim 1, wherein the antigen is a cell surface receptor.

7. The culture of claim 1, wherein the antigen is a G protein-coupled receptor.

8. The culture of claim 1, wherein the antigen is an ion channel.

9. A method for autologous immunization of a chicken, comprising:
making a culture of primary fibroblast cells of claim 1 using fibroblasts from the comb of a chicken; and
immunizing the chicken with the cells.

10. The method of claim 9, wherein the method comprises:
(a) obtaining a culture of primary fibroblast cells from the comb of a chicken;
(b) introducing an exogenous nucleic acid into the cells, wherein the exogenous nucleic acid provides for presentation or secretion of an antigen that is foreign to the chicken; and
(c) immunizing the chicken with the cells.

11. The method of claim 10, wherein the chicken is a newly hatched chicken.

12. The method of claim 10, further comprising obtaining polyclonal antibodies from the chicken after immunization.

13. The method of claim 10, further comprising obtaining B cells from the chicken after immunization.

14. An isolated B cell or hybridoma thereof, from a chicken made by the method of claim 10.

15. A culture of primary fibroblast cells derived from the comb of a chicken.
